(19) United States
(12) Patent Application Publication
(10) Pub. No.: US 2004/0086567 A1
(43) Pub. Date: May 6, 2004

(54) Bioequivalent composition of itraconazole and a hydrophilic polymer

(76) Inventor: Pawan Seth, Irvine, CA (US)

Correspondence Address: HARNESS, DICKEY & PIERCE, P.L.C., P.O. BOX 828, BLOOMFIELD HILLS, MI, 48303, US

(21) Appl. No.: 10/284,024

(22) Filed: Oct. 30, 2002

(51) Int. Cl. A61K 31/496; A61K 9/14

(52) U.S. Cl. 424/486; 424/488; 514/254.07

(57) ABSTRACT

The invention provides an itraconazole composition comprising itraconazole dispersed in a hydrophilic polymer, and, optionally, a surfactant, having a dissolution at 30 minutes between 10 and 40%, at 45 minutes between 25 and 80%, at 60 minutes between 60 and 95%, at 90 minutes at least 80%, as measured using the type II paddle method at 100 rpm according to the US Pharmacopoeia, in a dissolution medium comprised of 1000 ml HCl 0.1N. The invention also provides a process for manufacturing this composition.

### BACKGROUND OF THE INVENTION

The invention relates to an itraconazole composition having a bioequivalency dissolution, and to a process for preparing it.

Low-solubility pharmaceutical compounds often exhibit the drawback of insufficient dissolution in gastric fluids that prejudices obtaining a plasmatic concentration sufficient to achieve the therapeutical effects. Much work has been undertaken to achieve a sufficient plasmatic concentration necessary to reach the desired therapeutic effects. Addition of a surfactant is a simple way to enhance dissolution. It allows to raise wettability of otherwise poorly-wettable drugs and hence to increase the dissolution rate of the drug. This technique, rather old, has been improved over the last years.

EP-A-0330532 provides co-micronization of a poorly-soluble drug (fenofibrate) with a surfactant (sodium laurylsulfate). The result is better than that obtained with pure mixing of the components. WO-A-9831360 discloses fluidized bed granulation on an inert support of a suspension of a drug, where the suspension is in an aqueous solution comprising the hydrophilic polymer. Itraconazole is mentioned as a possible drug to which this invention may apply.

There is a need for an itraconazole that would be bioequivalent, when compared to the existing marketed compositions such as Sporanox®, and which would be easier to manufacture, compared to the existing compositions. None of the above documents teaches or suggests the instant invention.

### SUMMARY OF THE INVENTION

The invention provides an itraconazole composition comprising itraconazole dispersed in a hydrophilic polymer, having a dissolution at 30 minutes between 10 and 40%, at 45 minutes between 25 and 80%, at 60 minutes between 60 and 95%, at 90 minutes at least 80%, as measured using the type II paddle method at 100 rpm according to the US Pharmacopoeia, in a dissolution medium comprised of 1000 ml HCl 0.1N. According to one embodiment, the weight ratio polymer:itraconazole is lower than 1.9. According to one embodiment, the composition comprises an inert carrier covered with at least one layer containing said itraconazole dispersed in a hydrophilic polymer and, optionally, a surfactant, wherein the weight ratio polymer:itraconazole is lower than 1.9. Said ratio may be from 0.5 to 1.9, especially from 1 to 1.9.

The invention also provides a process for preparing a composition of the invention comprising the steps of:

(i) preparing a solution of itraconazole and polymer, and, optionally, said surfactant, in an organic solvent, wherein the weight ratio polymer:itraconazole is lower than 1.9; and

(ii) applying said solution onto an inert carrier.

### DETAILED DESCRIPTION

The invention is designed for itraconazole. The composition of the invention generally comprises the ingredients according to the following proportions: itraconazole: between 6 and 30 wt %; polymer: between 10 and 60 wt %; surfactant: between 0.1 and 3 wt % and inert carrier: between 20 and 80 wt %. The inventor has found that surprisingly, when the granule as defined below is placed in an aqueous medium, dissolution provides bioequivalency, compared to existing formulations, in the case the weight ratio polymer:itraconazole is lower than 1.9 (preferably equal to or lower than 1.85, more preferably equal to or lower than 1.8). Also, the inventor has found that the invention provides surprising results, when compared to prior art techniques, i.e. fluidized bed granulation with a suspension.

In the framework of this invention, the expression “inert carrier” means any excipient, generally hydrophilic, pharmaceutically inert, crystalline or amorphous, in a particulate form, not leading to a chemical reaction under the operating conditions employed, and which is preferably soluble in an aqueous medium, notably in a gastric acid medium. Examples of such excipients are derivatives of sugars, such as lactose, saccharose, hydrolyzed starch (malto-dextrine), saccharose, mannitol, microcrystalline cellulose, etc. Preferably inert spheroids are used, such as those disclosed in “sugar spheres, US Pharmacopea XXV”. These inert spheroids may be obtained by many processes, including extrusion of a water paste, followed by spheronization. These are available from Seppic, France or Werner, Germany. Mixtures are also suitable. The individual particle size of the inert hydrosoluble carrier can be, for example, between 50 and 500 microns, e.g. between 100 and 400 microns.

The expression “hydrophilic polymer” in the invention should be taken to mean any high molecular weight substance (greater, for example, than 300) having sufficient affinity towards water to dissolve therein and form a gel, allowing the final obtention of a film. Examples of such polymers are polyvinylpyrrolidone, polyvinylalcohol, hydroxypropylcellulose, hydroxymethylcellulose, hydroxypropylmethylcellulose, methylcellulose, ethylcellulose, gelatin, etc. or any other film-forming polymer known in the art (such as the polymers disclosed in “Handbook of Pharmaceutical excipients”, 2^{nd} Ed., 1994, American Pharmaceutical Association, Washington, ISBN 0 91730 66 8, by Wade A., Weller P J.). Polymer blends are also suitable. The preferred hydrophylic polymer is HPMC. The HPMC used in this invention is, for example, such that a 2% aqueous solution of the polymer at 20° C. has a viscosity between 0,5 and 50 cP, preferably between 2 and 25 cP.

The term “surfactant” is used in its conventional sense in this invention. Any surfactant is suitable, whether it be amphoteric, non-ionic, cationic or anionic. Examples of such surfactants are: sodium lauryl sulfate, monooleate, monolaurate, monopalmitate, monostearate or another ester of (polyoxyethylene) sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer®, etc. Mixtures of surfactants are also suitable. The preferred surfactant is the monostearate ester of (polyoxyethylene) sorbitane. Additional agent, known in the art can be used together with the drug and the polymer, albeit this is not preferred.

The composition according to the invention is prepared by a novel process comprising spraying a solution of itraconazole and a hydrophilic polymer and, optionally, a surfactant, onto the inert cores. The method according to the invention consists in using the fluidized bed granulation principle, but with specific starting materials, in order to arrive at an improved dissolution profile and thus, at elevated bioavailability. In particular, the invention employs a solution of itraconazole in a solution of a hydrophylic polymer and, optionally, a surfactant, where the solvent is an organic solvent. The solvent will solubilize both itraconazole and the polymer. This solvent can be a chlorinated solvent, such as methylene chloride (aka dichloromethane), or an alcane, such as hexane. The chlorinated solvent is preferred. It can be mixed with a co-solvent such as an alcohol, e.g. ethyl alcohol or iso-propyl alcohol. The amount of the co-solvent can be up to 50% by volume, preferably up to 40%, especially between 25 and 40%.

The fluidized-bed granulation technique is widely used in the pharmaceutical industry for preparing capsules or tablets. Conventionally, according to the prior art, a powder or a mixture of powders (itraconazole+excipients) is put into suspension in the fluidized bed in a granulator, and a solution containing a binder and, optionally, a surfactant, is sprayed onto this bed to form granules. The fluidized-bed granulation technique is well known to those skilled in the art and reference should be made to standard works such as for example “Die Tablette”, by Ritschel, Ed. Cantor Aulendorf, pages 211-212.

The invention, as has been indicated, comprises spraying a solution in an organic solvent of itraconazole with a hydrophilic polymer onto an inert carrier. Following granulation, the granule formed consists of crystals of, for example, lactose, which are isolated (or possibly agglomerated together by the spray solution) and particles of itraconazole and polymer adhering to the crystal surface. The granule could similarly be constituted of coated crystals, which are agglomerated, or even of such an agglomerate having received a coating. The granule obtained with the instant process can be broadly defined as comprising inert carrier particles, which are either isolated or agglomerated together, and particles of itraconazole in admixture with a hydrophilic polymer, adhering to the carrier particles surface.

The significant starting product is the solution of itraconazole. This solution is prepared by putting the itraconazole into solution in a solution comprising the hydrophylic polymer and, optionally, a surfactant, in solution in an organic solvent. If a surfactant is employed, it is put into solution in the solvent (beaker+magnetic or vane stirrer). Next, the hydrophylic polymer (HPMC) is dispersed, while stirring, in the solution previously obtained. While still stirring, the itraconazole is dispersed in the form of a fine shower into the above solution, to form a solution. The order of these steps can be reversed. In case a co-solvent is used, part of the components can first be dissolved (or otherwise solubilized) in this co-solvent prior to its admixing with the solvent.

The itraconazole concentration in the solution is generally from 1 to 15% by weight, preferably from 2 to 10%. The hydrophylic polymer concentration in the solution is generally from 1 to 25% by weight, preferably 3 to 20%. The surfactant concentration in the solution is generally from 0 to 5% by weight, preferably below 2%. The total components (incl. but not limited to the three above) content in the solution is from 3 to 20% by weight, preferably 5 to 15%. Further studies have shown that is possible to specifically adapt the amount of solvent used, so as to avoid at the same time sticking problems due to too high concentration (more than 20%) and excessive spray rate due to too large volumes (more than 20 g/minute/kg of carrier particles).

The granules thus obtained can, if desired, be provided with an outer coating or compressed into tablets, or form agglomerates. The outer coating is applied using conventional coating techniques such as coating in a pan or fluidized bed coater.

The granule may be available for the patient in many forms, including in a hard capsule or in a tablet form. When administered in a capsule form, the granules will generally be mixed with a lubricant. When administered in a tablet form, the granules will generally be mixed with a disintegrant and a lubricant. The granules will generally represent 50 to 90% of the final weight of the pharmaceutical composition (tablet or capsule).

Examples of disintegrants are cross-linked polyvinylpyrrolidone, sodium croscarmellose, sodium carboxymethyl starch, modified starch or unmodified starch. Examples of lubricants are magnesium stearate, sodium stearyl fumarate, glycerol behenate and talc. Flow enhancing agents (such as colloidal silica) may also be used. The excipients can be any one traditionally used in the art. For more details about these excipients, one can refer to the disclosure in “Handbook of pharmaceutical excipients”, American Pharmaceutical Association, 1994 ISBN 0 91730 66 8, by Wade A. and Weller P. When the granule obtained (whether subsequently coated or not) is compressed to form tablets, this step can be implemented using any conventional technique which is suitable, for example using alternating or rotating compressing equipment.

### EXAMPLES

The following examples illustrate the invention without limiting it.

### Example 1

The following formulations are prepared, where Ex. 1A is the prior art technique (suspension) while Ex. 1B is in accordance with the invention.

| | Amount (g) | |
|---|---|---|
| Compound | Ex. 1A | Ex. 1B |
| Itraconazole | 100 | 100 |
| HPMC | 160 | 180 |
| Sorbitan monostearate | 0 | 5 |
| Sodium lauryl sulfate | 5 | 0 |
| Sugar spheres 20-25 mesh | 196.75 | 195 |
| Methylene chloride | — | 1647 |
| Ethyl alcohol | — | 910 |
| Water | 937 | — |

Surfactant is dissolved in water (Ex. 1A) or alcohol (Ex. 1B). Itraconazole is suspended in water (Ex. 1A) or dissolved in methylene chloride (Ex. 1B). HPMC is dissolved in water (Ex. 1A) or alcohol (Ex. 1B). Suspension (Ex. 1A) or solution (Ex. 1B) is sprayed on the sugar spheres in a fluidized bed granulator (Glatt® GPCG1), equipped with a bottom injection system (Würster®). The following parameters are used.

| Inlet Temperature (° C.) | 45-48° C. |
|---|---|
| Product Temperature (° C.) | 40-45° C. |
| Air Volume (m³/h) | 100-130 |
| Atomization Pressure (bar) | 2.5-3.0 |
| Nozzle diameter (mm) | 1.0 |
| Pump Speed (g/min) | 8-13 |

Dissolution tests are carried out, in an apparatus according to the US Pharmacopea, XXIV, type II paddle, at 100 rpm, in a dissolution medium comprised of 1000 ml of HCl 0.1N.

The results (expressed in dissolved fraction in %) are the following.

| Time (min) | 0 | 15 | 30 | 45 | 60 | 90 |
|---|---|---|---|---|---|---|
| 1A | 0 | 10 | 15 | 15 | 15 | 15 |
| 1B | 0 | 5 | 29 | 72 | 87 | 91 |

Results clearly show that the release in the case of the invention is far different from the prior art.

### Example 2.

The following formulation is prepared.

| Compound | Amount (g) |
|---|---|
| Itraconazole | 100 |
| HPMC | 185 |
| Sorbitan monostearate | 5 |
| Sugar spheres 20-25 mesh | 195 |
| Methylene chloride | 1650 |
| Ethyl alcohol | 900 |

Surfactant is dissolved in alcohol under moderate stirring. HPMC then itraconazole are added to alcohol. Methylene chloride is then gradually added, resulting in solubilizing itraconazole and HPMC. The resulting solution is filtered on a mesh (100 mesh). It is then sprayed on the sugar spheres in fluidized bed granulator (Glatt® GPCG1), equipped with a top injection system (Top Spray®). The following parameters are used.

| Inlet Temperature (° C.) | 45-48° C. |
|---|---|
| Product Temperature (° C.) | 40-45° C. |
| Air Volume (m³/h) | 100-130 |
| Atomization Pressure (bar) | 2.5-3.0 |
| Nozzle diameter (mm) | 1.0 |
| Pump Speed (g/min) | 8-13 |

The resulting granules are filled in hard capsules (together with 15 g of silica as filler). These capsules are then subjected to a dissolution test identical to example 1.

The results (expressed in dissolved fraction in %) are the following.

| Time (min) | 0 | 15 | 30 | 45 | 60 | 90 |
|---|---|---|---|---|---|---|
| | 0 | 5 | 17 | 39 | 74 | 97 |

Results clearly show that the release in the case of the invention makes it bioequivalent to the existing Sporanox® product.

The invention claimed is:

1. An itraconazole composition comprising itraconazole dispersed in a hydrophilic polymer, and, optionally, a surfactant, having a dissolution at 30 minutes between 10 and 40%, at 45 minutes between 25 and 80%, at 60 minutes between 60 and 95%, at 90 minutes at least 80%, as measured using the type II paddle method at 100 rpm according to the US Pharmacopoeia, in a dissolution medium comprised of 1000 ml HCl 0.1N.

2. The composition of claim 1, wherein the weight ratio polymer:itraconazole is lower than 1.9.

3. The composition of claim 1, comprising an inert carrier covered with at least one layer containing said itraconazole dispersed in a hydrophilic polymer and, optionally, a surfactant, wherein the weight ratio polymer:itraconazole is lower than 1.9.

4. The composition of claim 1, wherein the hydrophilic polymer is chosen in the group consisting in polyvinylpyrrolidone, polyvinylalcohol, hydroxypropylcellulose, hydroxymethylcellulose, hydroxypropylmethylcellulose, methylcellulose, ethylcellulose, gelatin and mixtures thereof.

5. The composition of claim 4, wherein the hydrophilic polymer is hydroxypropylmethylcellulose.

6. The composition of claim 1, wherein the surfactant is present together with the hydrophilic polymer.

7. The composition of claim 6, wherein the surfactant is sodium lauryl sulfate; (polyoxyethylene) sorbitane monooleate, monolaurate, monopalmitate or monostearate; sodium dioctylsulfosuccinate, lecithin; stearylic alcohol; cetostearylic alcohol; cholesterol; polyoxyethylene ricin oil; polyoxyethylene fatty acid glyceride; poloxamer or a mixture thereof.

8. The composition of claim 7, wherein the surfactant is (polyoxyethylene) sorbitane monostearate.

9. The composition of claim 1, wherein itraconazole represents between 6 and 30 wt %.

10. The composition of claim 1, wherein the polymer represents between 10 and 60 wt %.

11. The composition of claim 1, wherein the surfactant represents between 0.1 and 3 wt %.

12. The composition of claim 3, wherein the inert carrier is an inert sugar sphere.

13. The composition of claim 3, wherein the inert carrier has a particle size between 50 and 500 microns.

14. The composition of claim 3, wherein the inert carrier represents between 20 and 80 wt %.

15. The composition of claim 1 in a capsule.

16. The composition of claim 1 compressed into a tablet.

17. A process for preparing a composition of claim 1 comprising the steps of:
(i) preparing a solution of itraconazole and polymer, and, optionally, said surfactant, in an organic solvent, wherein the weight ratio polymer:itraconazole is lower than 1.9; and
(ii) applying said solution onto an inert carrier.

18. The process of claim 17, wherein step (ii) comprises fluidized bed granulating.

19. The process of claim 17, wherein in step (i) the solvent is a chlorinated solvent, optionally in admixture with an alcohol.

20. The process of claim 19, wherein the solvent is methylene chloride, optionally in admixture with an alcohol.
